(19) United States
(12) Patent Application Publication
(10) Pub. No.: US 2006/0121052 A1
(43) Pub. Date: Jun. 8, 2006

(54) Recombinant vaccine from gE, gI, and gB proteins of the varicella-zoster virus for the treatment and prevention of multiple sclerosis

(76) Inventors: Julio Everardo Sotelo-Morales, Mexico, D. F. (MX); Maria Del Carmen Lara-Rodriguez, Mexico, D. F. (MX); Benjamin Pineda-Olvera, Mexico D. F. (MX)

Correspondence Address: SUGHRUE MION, PLLC, 2100 PENNSYLVANIA AVENUE, N.W., SUITE 800, WASHINGTON, DC, 20037, US

(21) Appl. No.: 11/264,167

(22) Filed: Nov. 2, 2005

(30) Foreign Application Priority Data
Nov. 3, 2004 (MX) PA/A/2004/010902

(51) Int. Cl. A61K 39/12

(52) U.S. Cl. 424/199.1; 424/204.1; 424/186.1

(57) ABSTRACT

Varicella-zoster virus belongs to the herpesvirus family and its main host are humans, producing 2 different diseases: varicella in children and young adults and zoster in elder or immunodepressed subjects. We reported in the scientific medical literature the unexpected finding that the role of varicella-zoster virus in the pathogeny of Multiple Sclerosis (Archives of Neurology 61: 529-532). This finding allows us to foresee the use of a vaccine against this virus with preventive and therapeutic ends for multiple sclerosis which eventually could also be applicable in the prevention of varicella and zoster. Currently the only vaccine used in humans is that produced by attenuated live varicella-zoster viruses, this latter feature thus avoiding its therapeutic use in multiple sclerosis, wherein the chronic disease is caused by periodic exacerbations of the virus which remains latent in the host, therefore by injecting an attenuated and viable virus the infection may be exacerbated and promote the very latency of the vaccine virus.

In our studies the most conspicuous genes of the varicella-zoster virus found in multiple sclerosis patients were the ones corresponding to the genes ORF31 (gB), ORF67 (gI) and ORF68 (gE). The recombinant vaccine which is the subject of this patent is built up by the proteins generated by these genes inserted in a plasmid vector of pNMT1-TOPO in order to transform Schizosaccharomyces pombe and thus obtaining the recombinant viral proteins which build up the vaccine.

This vaccine, by being made from recombinant viral proteins eliminates the risks associated to the use of vaccines from attenuated viable viruses. Likewise, the use of these recombinant viral proteins is specific and sensitive to serological tests for the diagnosis of infections caused by the varicella-zoster virus.

### BACKGROUND OF THE INVENTION

Varicella is a disease caused by the herpes virus which belongs to the herpesviridae family, Alfaherpesviridae subfamily. Varicella-zoster virus (VVZ) is a coated double stranded DNA virus that can produce at least two different diseases, on of them being varicella (clinical manifestation of primary infection) which is ubiquitous and highly contagious, and the other being herpes zoster, which appears as a consequence of the reactivation of VVZ which is latent in the nervous system and occurs usually in aged patients or immunocompromised subjects (1,2).

While varicella is a discrete and benign course disease characterized by fever and generalized vesicular rash, complications may occur including bacterial infections, pneumonia and encephalitis. Infection is more severe in adolescents and adults although about 90% if the cases occur in children (3,4), about 85% of primary varicella deaths occur in adults.

VVZ is obtained from primary varicella or herpes zoster patients through the direct contact with vesicular infected fluids or through respiratory secretion inhalations in spray form (5). Molecular studies suggest that the virus can be early transmitted by the respiratory pathway from 24 to 48 hours before the rash onset (6).

The virus incubating period is of 14 days (7), once the virus attacks the host's epithelial cells, it is phagocytated by mononuclear cells, replicating in them and being carried to the regional lymphoid nodes (8). Macrophages and monocytes can be infected, however there exist experiments demonstrating that the virus is lymphotropic, specifically to T cells (9). Four to six days after being exposed, an initial viremia is developed which is associated to mononuclear cells that carry the virus through the body to the endothelial reticulated cells (10). After the viral replication period, a second phase of high viremic titers initiates, resulting in virus dissemination. This second viremia stage by VVZ can be detected during the last 4 to 5 days of the incubating period and for few days after the onset of the typical rash (11). During this period infected mononuclear cells invade vascular endothelial cells, having access to skin tissue. After the primary infection is solved, VVZ travels centripetally and enters in a latent phase in the neural dorsal ganglia and can persist in this phase during all the subject's life (12).

The mechanism through which VVZ is carried from the mucosa to the lymphoid nodes is still unknown, however it is known to involve dendritic cells, glycoproteins on their capsid bind mannose receptors which are abundantly expressed by naive dendritic or non lymphoid cells present on peripheral tissues (13). Mature dendritic cells, which are located in the secondary lymphoid tissues act in vivo as potent antigen presenting cells and express high levels of MHC class I and II, CD40, coestimulatory molecules, CD80 and CD86, and adhesion molecules LFA-1, LFA-3, and ICAM-1 (13, 14).

VVZ infection in children is usually benign, but may be severe in elder adults, pregnant women and immunocompromised individuals, about 15% of the population mainly the elder can develop herpes zoster which is characterized by rash in a dermatoma distribution, followed by postherpetical pain (15).

Antibodies to CD86, HLA-DR, and other surface molecules expressed on these cells inhibit in vitro the sensitization of CD4+ virgin T cells, confirming their role in the adaptive immunity induction (16). Said cells are highly effective in presenting peptides to the virgin T cells (17, 18).

The immune response decreases after vaccination as age increases, however this response does not absolutely decays (19).

Wild type OKA VVZ was attenuated through in vitro passes on cell cultures by Takahashi, who discovered the efficacy of this virus as a vaccine (20, 21). After 15 years of experience in clinical trials, the attenuated live vaccine was approved in the United States in 1995 (19). Currently, the OKA varicella vaccine is the only approved vaccine for preventing the disease in humans. The wild type OKA vaccine was attenuated using an empirical process of growing in non human cells, taking advantage of the fact that the virus can be replicated in guinea pig embryo fibroblasts. After 11 passes on human lung fibroblasts, the OKA strain was passed 6 times on guinea pig embryo cells and transferred again to human lung fibroblasts. The subcutaneous inoculation of the OKA virus does not cause the disease in children, indicating that viremia does not occur or that it is subclinical, and the seroconversion is incremented (20). Due to the extreme restriction of hosts for VVZ limited to humans, animal models cannot be used to investigate whether this altered virulence pattern is also associated to decreases in its neurotropism. The vaccine against varicella-zoster is 85% protective against varicella and 97% protective against severe diseases (22).

The incidence of varicella in the United States before the introduction of this vaccine was of about 11000 hospitalizations and 100 deaths a year (23). Occasional complications include secondary bacterial infections, pneumonia, cerebellar ataxia encephalitis, transverse myelitis and death (15 24).

In the United States it was shown that from 1000 to 3000 pfu or more of the OKA vaccine increase adaptive immunity when it is subcutaneously administered to healthy children (25). A single dose is able to induce cellular and humoral immunity in more than 95% of the vaccinated recipients. Immunization increases IgG antibody levels as well as the response of cooperative and citotoxic T cells specific to VVZ (8). However the vaccine is less immunogenic in children over 12 years and in adults, but a 2 dose regime induces humoral and cellular responses similar to those obtained by vaccinating children under 12 years old with a single dose.

The main risk with the use of vaccines having live attenuated virus is their possible reversion to virulent, as well as their reactivation and dissemination. Krause shown in American children vaccinated with the OKA strain that the virus had the ability to reactivate, this was attained by examining the serum 6 weeks after the vaccination and thereafter in an annual format of 4631 children between 1 and 13 years old who received the vaccine before the same had its approval in the United States.

The vaccine against VVZ attenuated strain must not be administered to immunodeficient patients. It has been observed that patients with NKT cells deficiency are susceptible and may present respiratory problems and papulovesicular rash, apparently NK cells are one of the primary defense lines against VVZ (27). An important limitation to the conventional vaccine is that resistance of this vaccinal strain to the treatment with aciclovir was observed in immunosupressed children after being treated with antitumoral therapy (28). Further, VVZ reactivation has been observed in healthy children inducing varicella-zoster (29).

Varicella-zoster and Multiple Sclerosis

For many years many viruses have been associated with Multiple Sclerosis (EM) (30, 31). Epidemiological studies have suggested that VVZ is a good candidate (32, 33).

In a previous study of cases and controls performed by our team it was found that VVZ infection was the most significant risk factor in medical history of EM patients (34).

We have recently reported in international medical literature (Archives of Neurology 61: 529-532, 2004) the presence of VVZ in mononuclear cells of EM patients during exacerbation periods, the virus disappearing in the remission phase of the disease.

With this background facts associating VVZ to EM; we suggest the possibility that this virus is active in the etiopathogeny of the disease, a reason for which the search for a therapeutic or prevention alternative search might be the production of a recombinant vaccine from VVZ most immunogenic proteins and having no antigenic relation to the basic myelin protein. This latter aspect is important in that it has been shown that VVZ has amino acid sequences which molecularly mimic the basic myelin protein as well as some other viruses of the Herpesvirus family (35), this structural similarity between viral epitopes and the peptides themselves may raise an autoimmune response targeted by T cells, which is why the administration of a vaccine with live complete virus and solely attenuated as is the case of the vaccinal OKA strain may cause an exacerbation of the autoimmune response against the myelin basic protein used by Ross et al., which produced exacerbation of the symptoms in some multiple sclerosis patients who received the vaccine with the OKA strain (33).

Many immunogenic viral proteins may enhance the immune response against VVZ in such a manner that an optimal immune response or similar to the one obtained with the OKA vaccinal strain can be obtained, thus avoiding the possible risks of administering a live and attenuated virus vaccine.

### SUMMARY OF THE INVENTION

Recombinant gB, gR and gI proteins obtained from VVZ stimulate the immune response of mice immunized with such proteins, this response being similar to the one obtained by immunizing with the vaccinal strain indicating that in humans it may confer protection against the viral infection.

### BRIEF DESCRIPTION OF THE DRAWINGS

FIG. 1 shows the map of plasmid pNMT-TOPO.

FIG. 2 shows the amino acid sequence (SEQ ID NO:1) and polynucleotide sequence (SEQ ID NO:2) of the VVZ gB protein.

FIG. 3 shows the amino acid sequence (SEQ ID NO:3) and polynucleotide sequence (SEQ ID NO:4) of the VVZ gE protein.

FIG. 4 shows the amino acid sequence (SEQ ID NO:5) and polynucleotide sequence (SEQ ID NO:6) of the VVZ gI protein.

### DESCRIPTION OF AN EMBODIMENT

The cloned sequences coding for VVZ gE, gB and gI proteins in the plasmid pNMT1-TOPO, transforming a Schizosaccharomyces pombe (S. pombe) strain which methodology is disclosed in the following:

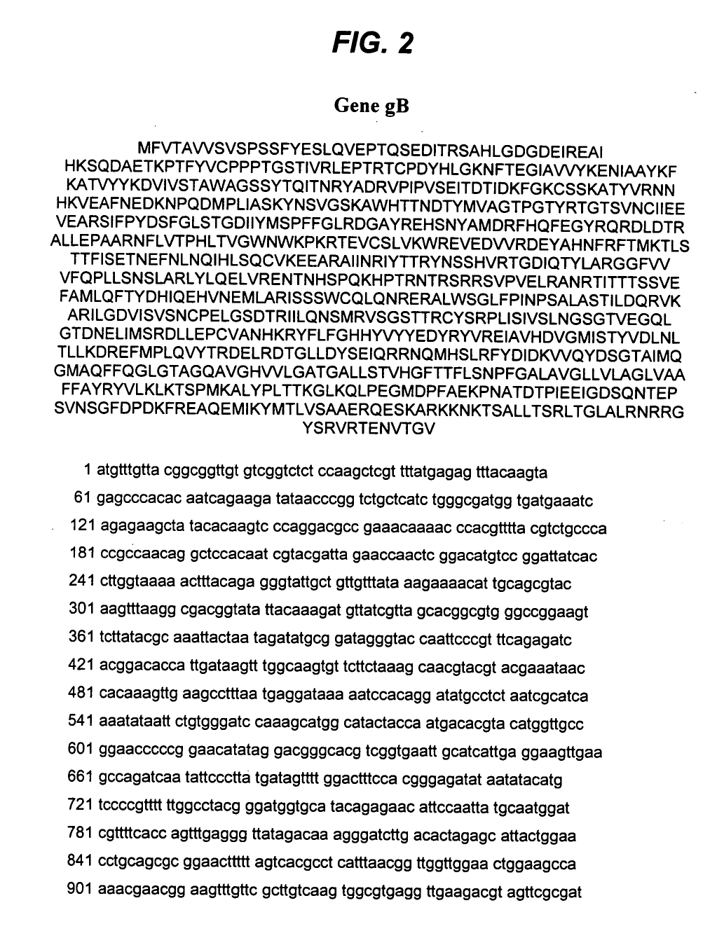

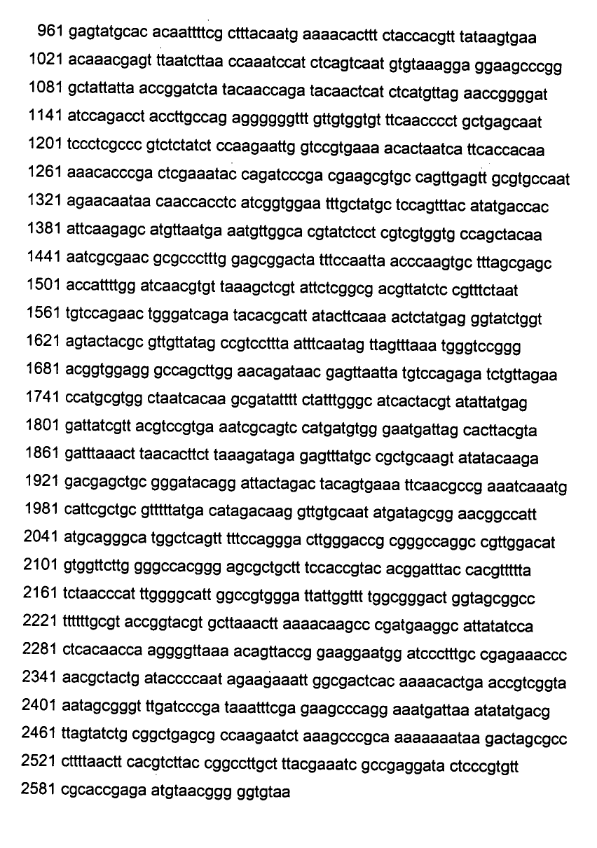

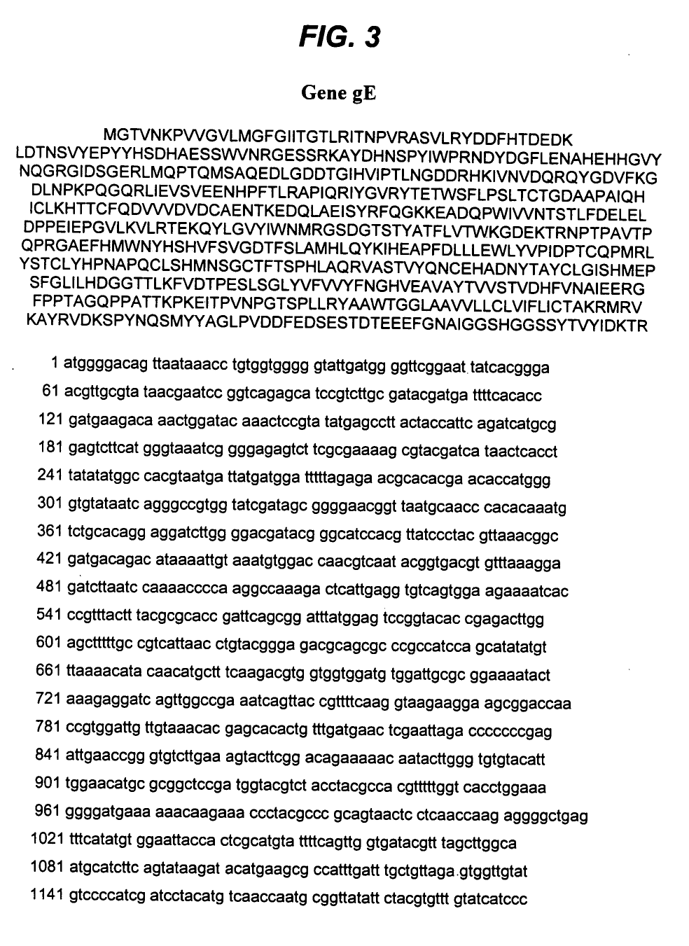

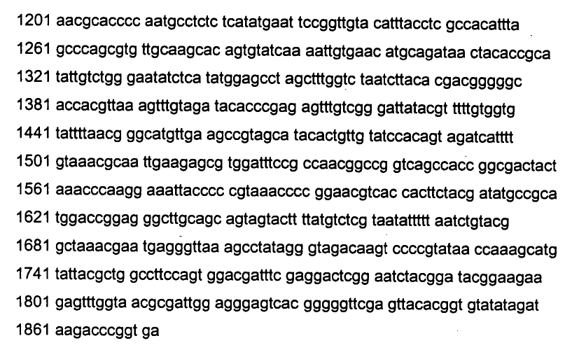

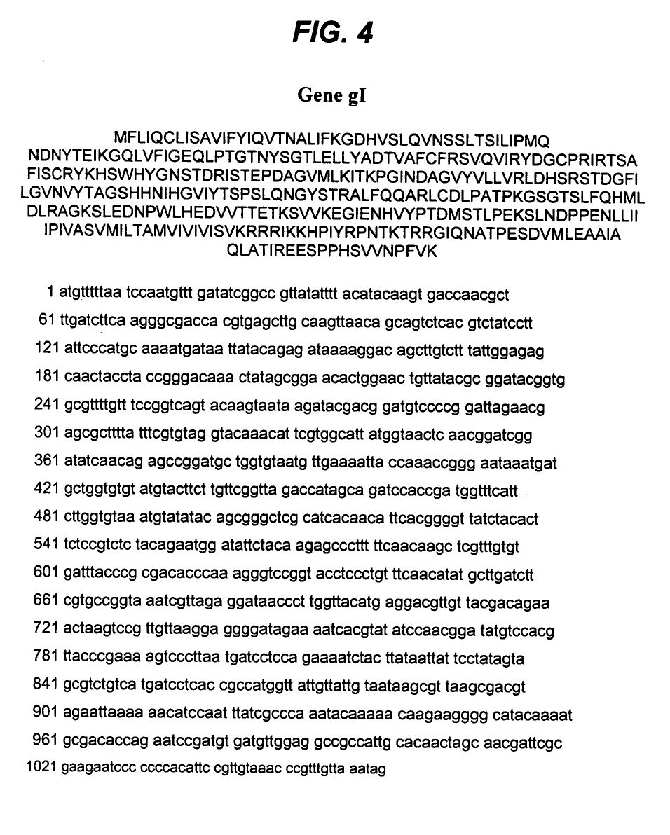

The coding sequences of VVZ gB (shown in FIG. 2), gE (shown in FIG. 3) and gI (shown in FIG. 4) proteins are obtained from the GenBank (www.ncbi.nih.gov) and the specific primers are designed using the DNA Man program, with this primers the DNA sequences are amplified by PCR.

DNA from which the desired sequences are amplified is extracted from the VVZ OKA strain. 200 μl VVZ are placed in a 1.5 ml eppendorf tube and adding 200 μl of lysis regulator with 2 mg/ml proteinase K, the sample is incubated overnight (12 hrs.). Once this time elapses 200 μl of chloroform:isoamyl alcohol (49:19) are added inversion mixed. Afterwards, they are centrifuged at 14000 rpm/15 min. at 4° C., the aqueous phase is transferred to another tube and the DNA is precipitated with 20 μL of 3M sodium acetate pH 5.2 and 2.5 volumes of 100% ethanol (about 500 μL). This is inversion mixed and kept at −20° C. for 30 min. or 15 min. at −70° C. Centrifugation takes place at 14000 rpm/15 min. and the supernatant is discarded, left dry by evaporation and resuspended in distilled water. Once the DNA is obtained it proceeds to the amplification of the desired genes by PCR.

### Experimental Design

DNA amplification of the genes coding for gB, gE and gI proteins by PCR.

DNA PCR

a) 5 μL of the DNA are used in a 5 μg/μL concentration diluted in water-DEPC (diethyl pyrocarbonate) plus 45 μL of the reaction mixture for PCR obtaining a final volume of 50 μL, as depicted in the following Table:

| | | |
|---|---|---|
| Reagent | Volume | Final Conc. |

| | | | | |
|---|---|---|---|---|
| Water-DEPC | 35.3 | μL | | |
| 10× PCR buffer | 5 | μL | 1× | |
| MgCl₂ 50 mM | 2.5 | μL | 0.5-4 | mM |
| DNTP's 10 mM Gibco BRL | 1.0 | μL | 200 | μM |
| “Primer” 3′ 20 μM | 0.5 | μL | 0.2-1 | μM |
| “Primer” 5′ 20 μM | 0.5 | μL | 0.2-1 | μM |
| Taq polimerase 5 U/μL Gibco BRL | 0.2 | μL | 1-2.5 | U/μL |
| cDNA | 5.0 | μL | | |

b) The reaction takes place in the thermocycler with the following features:

| | | | |
|---|---|---|---|
| 30 | cycles | Dissociation temperature | 94° C. |
| 45 | seconds | aligning temperature | 60° C. |
| 45 | seconds | extension temperature | 72° C. |
| 90 | seconds | 1 cycle | 72° C. |
| 7 | minutes | | |

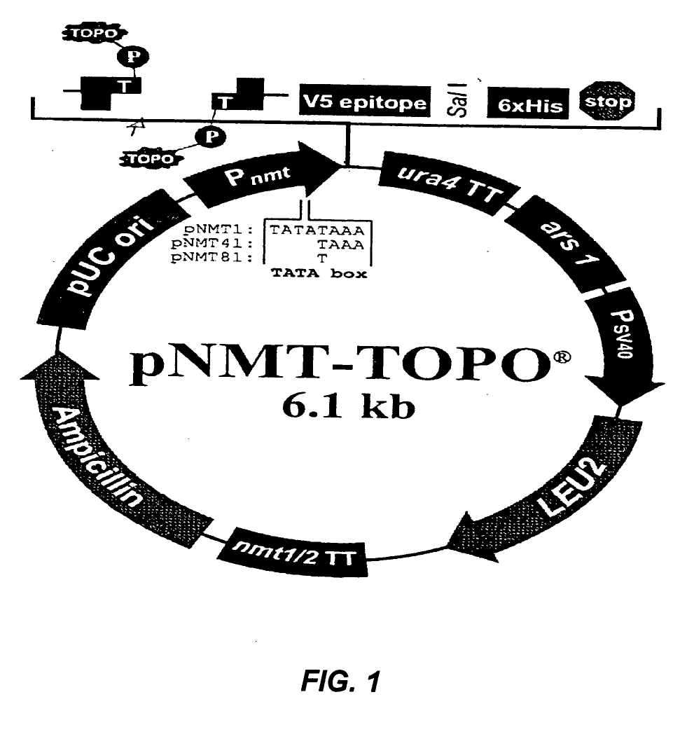

the PCR-amplified genes are cloned in a plasmid pNMT1-TOPO which restriction genetic map and cloning sequences are shown in FIG. 1. The vector is dissolved in Tris-HCl buffer with 10 ng/μL plasmid ratio in 5.0 of glycerol.

The PCR product is inserted in the plasmid vector, the vector of pNMT1-TOPO codes for a 6 histidine tag which expresses coupled to our interest proteins which are in the reading frame of said proteins. Once the plasmid pNM1-TOPO construct is obtained for the expression, it is ready for transforming S. pombe by electrophoresis and this way are the desired proteins obtained.

The transformation mixture is placed on Edinburgh Minimal Medium plates containing the relevant antibiotics, incubated overnight (16 hrs.) at 30° C. until the colonies are 1-2 mm diameter (master plates), once this time has elapsed the plates are removed from the incubator and the lids are carefully opened for 15-30 minutes in order to discard any possible generated condensation, and then the results are screened, the colonies having the vector+PCR insert are up to 85% and blue in color, the colonies having the vector alone will be white in color.

The transformed colonies are taken in 1.5 ml of culture media containing ampicillin (100 μg/ml) and Tiamine. A culture of 1.5 ml of a colony transformed with the empty plasmid pUC19, expressing the 6 histidine tag is also inoculated as a not induced control, the cultures are left under incubation overnight at 30° C., once the incubation has occurred 50 ml of EMM+T medium are inoculated. After this time the cells are obtained by centrifuging at 1500×g for 5 minutes. The supernatant is decanted and washed once with EMM medium, the cells are resuspended in 50 ml EMM, 500 μl aliquots are inoculated for stationary cultures in parallel in 2 vials of 100 ml EMM. Supplementing one or two of the cultures with 10 μM tiamine. They are incubated at 30° C. with stirring for 18 hours, collecting the cells for the screen: Centrifuging at 1500×g 5 min. at 4° C., the cells are resuspended in 1 ml TE1x+100 mM NaCl, centrifuged again, the cell pellet is resuspended in 1 ml TE1x+100 mM NaCl and transferred to a sterile microtube which is centrifuged 2 min. at maximum speed, the supernatant is removed and the cell pellets are stored at −80° C. until use.

The cells are lysed both thawed and fresh, resuspended in 500 μL TE1x+100 M NaCl, 400 μl of acid washed glass beads are added, disrupting the cells at maximum speed for 45 seg. in an agitator, placed on ice for 5 minutes and this process is repeated 5 times, they are centrifuged 2 min. at maximum speed, the supernatant is removed and transferred to a new tube; finally the protein titer is assessed using BSA as standard, this extracts are stored at −20° C. and analyzed by SDS-PAGE.

10 ml of LB medium containing 100 μg/ml ampicillin and 25 μg/ml kanamicin are inoculated in 50 ml vials, the vials growing overnight at 37° C. with stirring, after the incubation 50 ml of preheated medium (with antibiotics) is inoculated with 2.5 ml of the overnight cultures, allowing to grow at 37° C. with vigorous stirring until attaining an OD₆₀₀ of 0.5-0.7 (about 30-60 mins.). Thereafter the protein expression is induced with IPTG at a final concentration of 1 mM, then the cultures will grow for 4-5 hours harvesting the cells by centrifugation at 4000×g for 20 minutes.

10 ml of culture medium containing ampicillin (100 μg/ml) and kanamicin (25 μg/ml) are inoculated in a 50 ml vial, growing the culture at 37° C. overnight, with this culture 100 ml of preheated medium (with antibiotics) are inoculated together with 5 ml of the previously incubated culture and allowing to grow at 37° C. with vigorous stirring until reaching an OD₆₀₀ of 0.6 (30-60 minutes), 1 ml of the sample is collected just before the induction for its analysis by SDS-PAGE, the grown cells are centrifuged at 4000×g for 20 min. and thawed with liquid nitrogen.

20 ml of LB broth containing 100 μg/ml ampicillin and 25 μg/ml kanamicin are inoculated, thus growing it at 37° C. overnight with vigorous stirring, with this culture 1 liter of LB broth containing 100 μg/ml ampicillin and 25 μg/ml kanamicin 1:50 are inoculated with the not induced culture growing it at 37° C. overnight with vigorous stirring until attaining an OD₆₀₀ of 0.6, and a sample is taken to be analyzed by SDS-PAGE. Once the incubation time has elapsed the expression of the recombinant protein with IPTG at a final concentration of 1 mM is induced and the culture is incubated for 4-5 hours, a sample is taken for verifying the induction of the protein by SDS-PAGE, afterwards, the cells are collected by centrifuging at 4000×g for 20 minutes and stored by thawing them with liquid nitrogen.

For the immunization 120 Balb/c strain mice are used separated in 6 groups; group 1 are immunized with 5 μg of the gE recombinant protein emulsified with Freund's complete adjuvant; group 2 are immunized with 5 μg of the gB recombinant protein emulsified with Freund's complete adjuvant; group 3 are immunized with 5 μg of the gI recombinant protein emulsified with Freund's complete adjuvant; group 4 are immunized with 5 μg of the gB and gI recombinant proteins emulsified with Freund's complete adjuvant; group 5 are immunized with 5 μg of the gE, gB and gI recombinant proteins emulsified with Freund's complete adjuvant; group 6 (control) are immunized with 5 μg of the OKA vaccinal strain emulsified with Freund's complete adjuvant. 21 days after the first immunization a second immunization is applied emulsifying the antigens with Freund's incomplete adjuvant.

Blood samples are collected on days 21, 42, 63 and 84 post-immunization, and the mice are sacrificed on day 84 post-immunization to obtain their spleens.

The obtained recombinant proteins are screened by SDS-PAGE in 10% polyacrilamide gel. After electrophoresis the proteins are visualized by Coomassie blue staining and/or transferred to nitrocellulose membranes using a transblotting semi-dry system. The membranes are saturated for 30 minutes with 0.5% instagel in TBS-T (50 mM Tris HCl pH 7.5, 150 mM NaCl, 0.1% Tween 80), incubated with rabbit polyclonal serum hyperimmunized with VVZ vaccinal strain. The immunoreactive materials are detected using anti-rabbit antibodies conjugated to alkaline phosphatase.

To asses the humoral immunity the ELISA polystyrene plates are coupled with the VVZ recombinant proteins diluted 1:100 in carbonate buffer 0.1 M (pH 9.6) overnight at 4° C., washed 3 times with PBS containing polysorbate 20 at a concentration of 0.05%. Then, the sera of mice immunized with recombinant proteins are diluted in PBS-Polysorbate 20 1:10 by incubating at 4° C. overnight, once the incubation time has elapsed the plates are washed 3 times with PBS-Polysorbate 20 and a 1:100 dilution of mice anti IgM or mice anti IgG in PBS-Polysorbate produced in alkaline phosphatase-conjugated rabbit are added, the plates are incubated at 37° C. for 90 minutes, and then washed with PBS polysorbate. The color is developed with p-nitrophenyl phosphate in diethanolamine buffer (pH 9.8) for 30 min. at 37° C., the reaction is stopped with 3M sodium hydroxide and read at 420 nm in an ELISA spectrophotometer.

In order to asses cellular immunity a spleen cell culture is performed of the mice immunized with the VVZ recombinant vaccines in 24 well plates with 4-6×10⁶ cells per well in AIM-V medium, which are stimulated with the recombinante proteins of the VVZ vaccinal strain and with 24 μg/ml of PMA-Ionomicine and they are incubated at 37° C. in a wet atmosphere of 5% CO₂, 2 days after the culture 10 μl BrdU are added until getting a final concentration of 60 μM. The cells are incubated for 5-6 hours at 37° C. and 5% CO₂. At the end of the incubation 400 μl of EDTA solution are added to each well and the cells are moved to Falcon tubes vortexing them for 15 seconds at a high rate in order to then incubate the slanted tubes for 15 minutes. At room temperature and once the incubation time has elapsed, 400 μl of cold PBS are added and vigorously mixed in vortex, afterwards centrifuging at 1200 rpm for 10 minutes the supernatant is discarded and 3 ml of a lysis solution are added 1× to each tube in order to incubate them afterwards for 10-12 minutes at room temperature. Once incubation time has elapsed it is centrifuged at 1200 rpm for 10 minutes, the supernatant is decanted and 2 ml of the washing solution (PBS) are added, 2 ml of the permeating solution are added 1× and they are incubated for 10 minutes at room temperature, then, it is washed with 2 ml PBS and centrifuged at 1200 rpm for 10 minutes, the supernatant is disposed and the sample is partitioned in 5 tubes marked as γ-FITC/γ-PE and CD2/BrdU, CD4/BrdU, CD8/BrdU and NK/BrdU, the samples are centrifuged at 1200/rpm for 10 min. and the supernatant is disposed, the cells are resuspended in the remaining volume, then 5 μl of the isotype control are added to the tube marked as γ-FITC/γ-PE, 5 μl anti-CD2-PE plus 15 μl anti BrdU-DNAse-FITC to the tube marked as CD2/BrdU, 5 μl anti-CD4-PE plus 15 μl anti BrdU-DNAse-FITC to the tube marked as CD4/BrdU, 5μ anti-CD8-PE plus 15 μl anti BrdU-DNAse-FITC to the tube marked as CD8/BrdU, 5 μl anti-NK-PE plus 15 μl anti BrdU-DNAse-FITC to the tube marked as NK/BrdU, incubated 30 min. in the dark, washed with 2-3 ml PBS and 500 μl of 1% paraformaldehyde are added. The samples are kept under refrigeration until their analysis in the flux citometer.

The experimentally obtained results enable us to foresee that this recombinant vaccine can be effective in the prevention and treatment of Multiple Sclerosis in humans.

1.- Use of recombinant proteins from gE, gI and gB genes of varicella-zoster virus in the manufacture of a medicament for treating and preventing diseases and disorders related to the varicella-zoster virus in mammals.

2.- Use of recombinant proteins from gE, gI and gB genes of varicella-zoster virus according to claim 1, wherein the disease is Multiple Sclerosis in humans.

3.- Use of recombinant proteins from gE, gI and gB genes of varicella-zoster virus according to claim 1, wherein the disease is varicella or herpes in humans.

4.- Use of recombinant proteins from gE, gI and gB genes of varicella-zoster virus in the manufacture of a diagnosis reagent for the serological diagnosis of varicella-zoster virus infection in mammals.


SEQUENCE LISTING (6 sequences)

SEQ ID NO: 1 - protein, 868 aa, Varicella zoster
MFVTAVVSVSPSSFYESLQVEPTQSEDITRSAHLGDGDEIREAIHKSQDAETKPTFYVCPPPTGSTIVRLEPTRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLSTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEHVNEMLARISSSWCQLQNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 2 - DNA, 2607 bp, Varicella zoster
atgtttgttacggcggttgtgtcggtctctccaagctcgttttatgagagtttacaagtagagcccacacaatcagaagatataacccggtctgctcatctgggcgatggtgatgaaatcagagaagctatacacaagtcccaggacgccgaaacaaaacccacgttttacgtctgcccaccgccaacaggctccacaatcgtacgattagaaccaactcggacatgtccggattatcaccttggtaaaaactttacagagggtattgctgttgtttataaagaaaacattgcagcgtacaagtttaaggcgacggtatattacaaagatgttatcgttagcacggcgtgggccggaagttcttatacgcaaattactaatagatatgcggatagggtaccaattcccgtttcagagatcacggacaccattgataagtttggcaagtgttcttctaaagcaacgtacgtacgaaataaccacaaagttgaagcctttaatgaggataaaaatccacaggatatgcctctaatcgcatcaaaatataattctgtgggatccaaagcatggcatactaccaatgacacgtacatggttgccggaacccccggaacatataggacgggcacgtcggtgaattgcatcattgaggaagttgaagccagatcaatattcccttatgatagttttggactttccacgggagatataatatacatgtccccgttttttggcctacgggatggtgcatacagagaacattccaattatgcaatggatcgttttcaccagtttgagggttatagacaaagggatcttgacactagagcattactggaacctgcagcgcggaactttttagtcacgcctcatttaacggttggttggaactggaagccaaaacgaacggaagtttgttcgcttgtcaagtggcgtgaggttgaagacgtagttcgcgatgagtatgcacacaattttcgctttacaatgaaaacactttctaccacgtttataagtgaaacaaacgagtttaatcttaaccaaatccatctcagtcaatgtgtaaaggaggaagcccgggctattattaaccggatctatacaaccagatacaactcatctcatgttagaaccggggatatccagacctaccttgccagaggggggtttgttgtggtgtttcaacccctgctgagcaattccctcgcccgtctctatctccaagaattggtccgtgaaaacactaatcattcaccacaaaaacacccgactcgaaataccagatcccgacgaagcgtgccagttgagttgcgtgccaatagaacaataacaaccacctcatcggtggaatttgctatgctccagtttacatatgaccacattcaagagcatgttaatgaaatgttggcacgtatctcctcgtcgtggtgccagctacaaaatcgcgaacgcgccctttggagcggactatttccaattaacccaagtgctttagcgagcaccattttggatcaacgtgttaaagctcgtattctcggcgacgttatctccgtttctaattgtccagaactgggatcagatacacgcattatacttcaaaactctatgagggtatctggtagtactacgcgttgttatagccgtcctttaatttcaatagttagtttaaatgggtccgggacggtggagggccagcttggaacagataacgagttaattatgtccagagatctgttagaaccatgcgtggctaatcacaagcgatattttctatttgggcatcactacgtatattatgaggattatcgttacgtccgtgaaatcgcagtccatgatgtgggaatgattagcacttacgtagatttaaacttaacacttcttaaagatagagagtttatgccgctgcaagtatatacaagagacgagctgcgggatacaggattactagactacagtgaaattcaacgccgaaatcaaatgcattcgctgcgtttttatgacatagacaaggttgtgcaatatgatagcggaacggccattatgcagggcatggctcagtttttccagggacttgggaccgcgggccaggccgttggacatgtggttcttggggccacgggagcgctgctttccaccgtacacggatttaccacgtttttatctaacccatttggggcattggccgtgggattattggttttggcgggactggtagcggccttttttgcgtaccggtacgtgcttaaacttaaaacaagcccgatgaaggcattatatccactcacaaccaaggggttaaaacagttaccggaaggaatggatccctttgccgagaaacccaacgctactgataccccaatagaagaaattggcgactcacaaaacactgaaccgtcggtaaatagcgggtttgatcccgataaatttcgagaagcccaggaaatgattaaatatatgacgttagtatctgcggctgagcgccaagaatctaaagcccgcaaaaaaaataagactagcgcccttttaacttcacgtcttaccggccttgctttacgaaatcgccgaggatactcccgtgttcgcaccgagaatgtaacgggggtgtaa

SEQ ID NO: 3 - protein, 623 aa, Varicella zoster
MGTVNKPVVGVLMGFGIITGTLRITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 4 - DNA, 1872 bp, Varicella zoster
atggggacagttaataaacctgtggtgggggtattgatggggttcggaattatcacgggaacgttgcgtataacgaatccggtcagagcatccgtcttgcgatacgatgattttcacaccgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggactcggaatctacggatacggaagaagagtttggtaacgcgattggagggagtcacgggggttcgagttacacggtgtatatagataagacccggtga

SEQ ID NO: 5 - protein, 354 aa, Varicella zoster
MFLIQCLISAVIFYIQVTNALIFKGDHVSLQVNSSLTSILIPMQNDNYTEIKGQLVFIGEQLPTGTNYSGTLELLYADTVAFCFRSVQVIRYDGCPRIRTSAFISCRYKHSWHYGNSTDRISTEPDAGVMLKITKPGINDAGVYVLLVRLDHSRSTDGFILGVNVYTAGSHHNIHGVIYTSPSLQNGYSTRALFQQARLCDLPATPKGSGTSLFQHMLDLRAGKSLEDNPWLHEDVVTTETKSVVKEGIENHVYPTDMSTLPEKSLNDPPENLLIIIPIVASVMILTAMVIVIVISVKRRRIKKHPIYRPNTKTRRGIQNATPESDVMLEAAIAQLATIREESPPHSVVNPFVK

SEQ ID NO: 6 - DNA, 1065 bp, Varicella zoster
atgtttttaatccaatgtttgatatcggccgttatattttacatacaagtgaccaacgctttgatcttcaagggcgaccacgtgagcttgcaagttaacagcagtctcacgtctatccttattcccatgcaaaatgataattatacagagataaaaggacagcttgtctttattggagagcaactacctaccgggacaaactatagcggaacactggaactgttatacgcggatacggtggcgttttgtttccggtcagtacaagtaataagatacgacggatgtccccggattagaacgagcgcttttatttcgtgtaggtacaaacattcgtggcattatggtaactcaacggatcggatatcaacagagccggatgctggtgtaatgttgaaaattaccaaaccgggaataaatgatgctggtgtgtatgtacttcttgttcggttagaccatagcagatccaccgatggtttcattcttggtgtaaatgtatatacagcgggctcgcatcacaacattcacggggttatctacacttctccgtctctacagaatggatattctacaagagccctttttcaacaagctcgtttgtgtgatttacccgcgacacccaaagggtccggtacctccctgtttcaacatatgcttgatcttcgtgccggtaaatcgttagaggataacccttggttacatgaggacgttgttacgacagaaactaagtccgttgttaaggaggggatagaaaatcacgtatatccaacggatatgtccacgttacccgaaaagtcccttaatgatcctccagaaaatctacttataattattcctatagtagcgtctgtcatgatcctcaccgccatggttattgttattgtaataagcgttaagcgacgtagaattaaaaaacatccaatttatcgcccaaatacaaaaacaagaaggggcatacaaaatgcgacaccagaatccgatgtgatgttggaggccgccattgcacaactagcaacgattcgcgaagaatcccccccacattccgttgtaaacccgtttgttaaatag